(19) United States
(12) Patent Application Publication
(10) Pub. No.: US 2026/0175005 A1
(43) Pub. Date: Jun. 25, 2026

(54) BLOOD PUMP

(71) Applicant: Abiomed Europe GmbH, Aachen (DE)

(72) Inventors: Wolfgang Kerkhoffs, Aachen (DE); Marius Grauwinkel, Aachen (DE); Ellen Keysselitz, Aachen (DE)

(73) Assignee: Abiomed Europe GmbH, Aachen (DE)

(21) Appl. No.: 18/729,315

(22) PCT Filed: Jan. 25, 2023
(86) PCT No.: PCT/EP2023/051804

§ 371 Date: Jul. 16, 2024

(30) Foreign Application Priority Data
Jan. 28, 2022 (EP) 22 153 922.4

(51) Int. Cl. A61M 60/13; A61M 60/226; A61M 60/422

(52) U.S. Cl. CPC A61M 60/13; A61M 60/226; A61M 60/422

(57) ABSTRACT

The present invention relates to a blood pump (10), in particular intravascular blood pump. The blood pump (10) comprise a pump housing (12) having a blood flow inlet (14) and a blood flow outlet (16) connected by a passage (18), an impeller (20) disposed in said pump housing (12), and a drive unit (26) configured to drive the impeller (20). The pump housing (12) comprises a drive unit casing (22) and the drive unit (26) is disposed within the drive unit casing (22), wherein the drive (26) unit comprises a stator (66) and an insulation assembly (68), and wherein the insulation assembly (68) is configured to prevent electrical leakage.

The present invention relates to a blood pump. In particular, the present invention relates to an intravascular blood pump for percutaneous insertion into a patient's blood vessel, to support a blood flow in a patient's blood vessel. The blood pump may also be an intracardiac blood pump or any other kind of ventricular assist device.

### BACKGROUND OF THE INVENTION

Various blood pumps are known from the prior art, e.g. axial blood pumps, centrifugal (i.e. radial) blood pumps or mixed-type blood pumps, where the blood flow is caused by axial forces as well as by radial forces. Such blood pumps may be introduced into the heart of a patient to support the blood flow from the heart into an artery e.g., the aorta. The blood pump may be introduced percutaneously during a cardiac procedure through the vascular system, such as by a catheterization procedure. After the blood pump has been placed, blood is unloaded by the blood pump from the left ventricle into the aorta to restore adequate systemic blood flow. Therefore, a blood pump typically comprises of a pump housing having a blood flow inlet and a blood flow outlet connected by a passage, a pump element in form of an impeller disposed in said pump housing and a drive unit configured to drive the impeller. An according blood pump is known from e.g., WO 2021/043776 A1.

The blood pump disclosed in WO 2021/043776 A1 comprises a drive unit configured to drive the impeller contact free. Therefore, the impeller is magnetically coupled to a stator in that the impeller comprises magnets which are disposed adjacent to electrically magnetized zones in the stator. Based on the attractive force between the magnets of the impeller and the magnetized zones in the stator, rotation can be transmitted to the impeller. In particular, a rotating magnetic field is established within the stator which rotates the impeller in that a control unit applies appropriate voltage to the stator in a controlled manner.

However, under certain operating conditions unwanted electrical leakage or electrical breakdown may occur between the stator and the pump housing. Said electrical leakage may impair the efficiency of the drive unit and hence, of the blood pump. Further, this may lead to a malfunction of the blood pump and consequent dangerous situations for the patient. Hence, it is an objective of the invention to improve the functional stability and efficiency of the blood pump.

### SUMMARY OF THE INVENTION

The blood pump according to the present invention may correspond to the aforementioned blood pump. Hence, the blood pump may be an intravascular blood pump or an intracardiac blood pump. According to a first aspect, the blood pump comprises a pump housing having a blood flow inlet and a blood flow outlet connected by a passage, a pump element, in particular an impeller, and a drive unit configured to drive the impeller. The impeller is disposed the pump housing. The pump housing comprises a drive unit casing and the drive unit is disposed within the drive unit casing. The drive unit comprises a stator and an insulation assembly, and the insulation assembly is configured to prevent electrical leakage.

The insulation assembly may comprise several insulation members specifically placed between components of the blood pump to inhibit electrical flow between the components. Thus, this greatly reduces the risk of electrical leakage and hence, of an impaired functional stability or efficiency of the blood pump.

Preferably, the insulation assembly comprises a spacer assembly configured to space the stator from the drive unit casing so as to inhibit contact between the stator and the drive unit casing. The spacer assembly may comprise a spacer configured to radially space the stator from an inner surface of the drive unit casing, in particular from an inner peripheral surface of the drive unit casing. The spacer may be provided as a backring. The spacer inhibits direct contact between the stator and the inner surface of the drive unit casing and thus, greatly minimizes electrical leakage between the stator and the drive unit casing.

Preferably, the stator has a catheter-side end pointing away from the impeller. The spacer may be ring-shaped and may have a tubular portion. The tubular portion may extend at least partially circumferentially. The spacer may be disposed at the catheter-side end of the stator so that the stator is partially disposed radially inside of the tubular portion of the spacer. Accordingly, the spacer may be firmly secured to the stator. In this connection, it has to be mentioned that the terms “at least partially” or “partially” as used herein mean both partial and entirely or complete respectively.

Preferably, the tubular portion of the spacer abuts against the inner surface of the drive unit casing, in particular against the inner peripheral surface of the drive unit casing. Thus, there is no direct contact between the stator and the drive unit casing which might otherwise cause electrical leakage.

Preferably, the stator comprises a backplate contacting the catheter-side end of the stator. The backplate may be disposed radially inside of the tubular portion of the spacer. The back plate enhances a magnetic flux of the stator, which allows for a reduction of the overall size of the blood pump. Preferably, the back plate comprises a suitable material or is composed of a suitable material, in particular a soft magnetic material, such as electrical steel or a suitable alloy, preferably cobalt steel. Cobalt steel has the highest magnetic permeability and highest magnetic saturation flux density amongst all conceivable electrical steels. Accordingly, the usage of cobalt steel allows for a reduced size of the blood pump, as the respective components composed of cobalt steel can be reduced in size compared to usage of other electrical steel.

Preferably, the drive unit further comprises a printed circuit board. The printed circuit board may at least partially be disposed radially inside of the spacer assembly. The printed circuit board is part of a control unit that applies appropriate voltage to the stator in a controlled manner to cause a rotating magnetic field and to thus drive the impeller.

Preferably, the insulation assembly comprises a frontplate configured to space the stator from the drive unit casing so as to inhibit contact between the stator and the drive unit casing. In particular, the frontplate inhibits contact between the stator and an inner surface of the drive unit casing, preferably between the stator and the inner peripheral surface of the drive unit casing. This further reduces the risk of electrical leakage between the stator and the drive unit casing. The frontplate may be considered as a part of the spacer assembly.

Preferably, the stator comprises a plurality of posts and coil windings disposed about the posts. The frontplate may have a central portion and a ring-shaped outer portion. A plurality of frontplate legs may extend between the central portion and the ring-shaped outer portion. The frontplate legs may be configured to circumferentially space each of the plurality of posts from an adjacent post. The plurality of posts may be partially disposed radially inwardly of the ring-shaped outer portion. The posts act as a magnetic core and are made of a suitable material, in particular a soft magnetic material, such as electrical steel or a suitable alloy, such as cobalt steel. The posts are preferably composed of the identical material as the backplate to enhance the magnetic flux. In addition, the frontplate legs warrant for a correct positioning of the plurality of posts relative to each other and relative to the drive unit casing, and aligns the posts and protects the coil windings. In particular, the radial clearance between the inner surface of the drive unit casing and the coil windings is set via the frontplate.

Preferably, the stator comprises a backbone having radially extending backbone legs. The backbone legs are preferably configured to circumferentially space each of the plurality of posts from an adjacent post. The backbone may be star-shaped. The backbone is made of a suitable material, in particular a soft magnetic material, such as electrical steel or a suitable alloy, such as cobalt steel. Preferably, the backbone is made of the identical material as the plurality of posts and the backplate.

The backplate and the plurality of posts may be integrally formed as a one-piece unitary member. This facilitates the assembly of the stator.

Preferably, the insulation assembly comprises a plurality of shrink elements, wherein each of the plurality of posts may be at least partially surrounded by one of the shrink elements. The shrink elements may be shrink sleeves. In particular, the shrink elements may be heat shrink elements. The shrink sleeves electrically separate each of the plurality of posts from the respective coil winding surrounding the post. This further reduces the risk of electrical leakage and additionally protects the coil windings in case an optional coil winding cover is broken.

Preferably, the shrink elements comprise polyester or are composed of polyester. Polyester has a good biocompatibility and further is a non-electrical conductive material.

Preferably, the stator has an impeller-side end pointing towards the impeller. The insulation assembly may further comprise a frontsheet. The frontsheet may cover the impeller-side end of the stator so as to inhibit contact between the stator and the drive unit casing. In particular, the frontsheet may inhibit contact between the stator and the drive unit casing in the axial direction. This further greatly reduces the risk of electrical leakage between the stator and the drive unit casing.

Preferably, the insulation assembly is at least partially composed of a non-electrical conductive material, wherein the insulation assembly is preferably entirely composed of the non-electrical conductive material. The non-electrical conductive material is preferably also a non-magnetizable material. Preferably, the non-electrical conductive material is a thermoplastic material, preferably a polyaryletherketone, wherein the non-electrical conductive material is preferably polyetheretherketone (PEEK). PEEK has a high biocompatibility and further greatly insulates the concerned components from each other.

Preferably, the drive unit casing is made of titanium or titanium alloy.

Preferably, an inner surface of the drive unit casing is at least partially coated with a non-electrical conductive coating, in particular diamond-like carbon (DLC). The inner surface may be the inner peripheral surface of the drive unit casing. As DLC is a non-electrical conductive material this further reduces the risk of electrical leakage. In addition, it is also possible to waive the insulation assembly partly or entirely and to coat the inner surface of the drive unit casing at least partially with DLC to inhibit electrical leakage between the stator and the drive unit casing. In this regard, direct contact between the stator and the drive unit casing may be allowed.

Preferably, the stator is at least partially surrounded by a shrink element circumferentially and/or the stator is at least partially coated with a non-electrical conductive coating. The shrink element may be a shrink sleeve and may in particular be a heat shrink element. Preferably, the shrink elements comprise polyester or are composed of polyester. In addition or as an alternative, the plurality of posts and/or the backplate and/or the backbone may be partially or entirely coated with a non-conductive material, for example diamond-like carbon. In addition, it is also possible to waive the insulation assembly partly or entirely and to provide the shrink element and/or the non-electrical conductive coating to inhibit electrical leakage between the stator and the drive unit casing. In this regard, direct contact between the stator and the drive unit casing may be allowed. In addition, a DLC coating as described above may be applied to the inner surface of the drive unit casing.

### BRIEF DESCRIPTION OF THE DRAWINGS

The foregoing summary as well as the following detailed description of preferred embodiments will be better understood when read in conjunction with the appended drawings. For the purpose of illustrating the present disclosure, reference is made to the drawings. However, the scope of the disclosure is not limited to the specific embodiments disclosed in the drawings.

In the drawings:

FIG. 1 shows a schematic perspective view of a blood pump,

FIG. 2 shows another schematic perspective view of the blood pump of FIG. 1,

FIG. 3 is a detailed schematic view of a catheter attachment portion of the blood pump of FIG. 1,

FIG. 4 is a schematic side view of the blood pump of FIG. 1,

FIG. 5 is a partial schematic cross-sectional view of the blood pump of FIG. 1,

FIG. 6 is a schematic side view of an impeller of the blood pump of FIG. 1,

FIG. 7 is a schematic back view of the impeller of FIG. 6,

FIG. 8 is a schematic back view of an alternative embodiment of an impeller,

FIG. 9 is a schematic cross-sectional view of a drive unit casing and a drive unit of the blood pump of FIG. 1,

FIG. 10 is a schematic exploded view of a stator and an insulation assembly of the blood pump of FIG. 1,

FIG. 11 is a schematic cross-sectional view of an alternative embodiment of an impeller supporting portion of the drive unit casing of FIG. 9,

FIG. 12 is a schematic detail of a pivot bearing,

FIG. 13 is a schematic detail of an alternative embodiment of a pivot bearing,

FIG. 14 is a schematic detail of a further alternative embodiment of a pivot bearing,

FIG. 15 is a schematic detail of a further alternative embodiment of a pivot bearing,

FIG. 16 is a schematic detail of a second pivot bearing member,

FIG. 17 is a schematic detail of an alternative embodiment of a second pivot bearing member,

FIG. 18 is a schematic detail of a further alternative embodiment of second pivot bearing member,

FIG. 19 is a schematic detail of a further alternative embodiment of a pivot bearing as a cross section,

FIG. 20 is the view of FIG. 19 with the impeller rotated by 90°,

FIG. 21 is a schematic detail of the first pivot bearing member and the second pivot bearing member of FIG. 19,

FIG. 22 is a schematic detail of a radial bearing,

FIG. 23 is a further schematic detail of the radial bearing, and

FIG. 24 is a schematic perspective view of a second radial bearing member of the radial bearing.

### DETAILED DESCRIPTION

### Blood Pump and Pump Housing

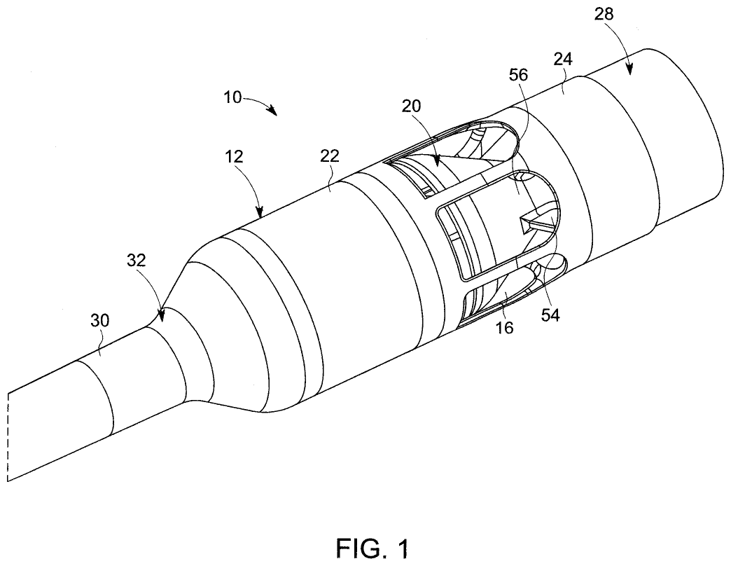

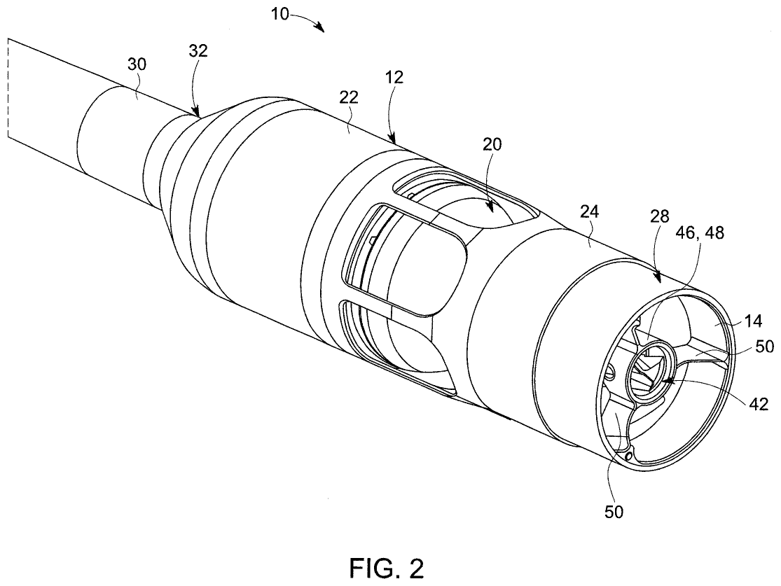

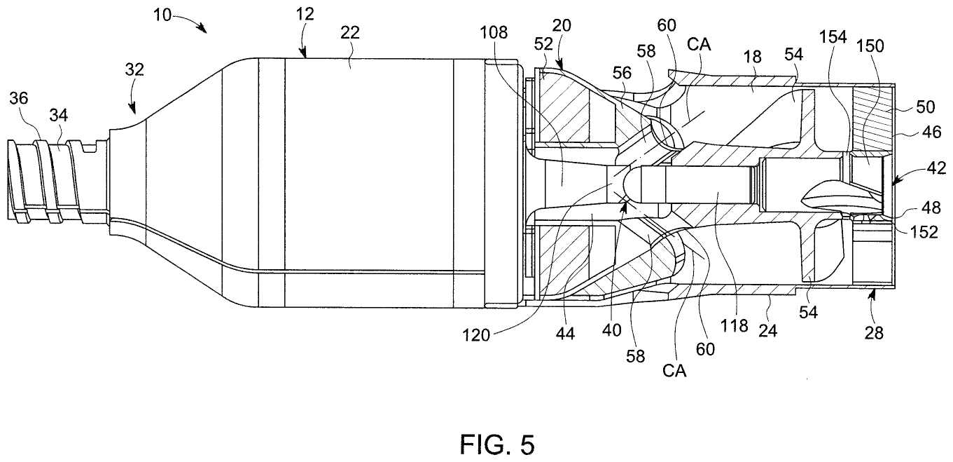

Referring initially to FIGS. 1, 2 and 4, schematic perspective views and a schematic side view of a blood pump 10 are illustrated. In this embodiment, the blood pump 10 is an intravascular blood pump, also known as a catheter pump. The blood pump 10 comprises a pump housing 12 having a blood flow inlet 14 and a blood flow outlet 16 connected by a passage 18 (see e.g., FIG. 5). Here, the blood flow outlet 16 is composed of a plurality of evenly distributed openings along the circumference of the pump housing 12. The pump housing 12 comprises of a drive unit casing 22 and an impeller casing 24, and an impeller 20 which is disposed in the pump housing 12, namely in the impeller casing 24.

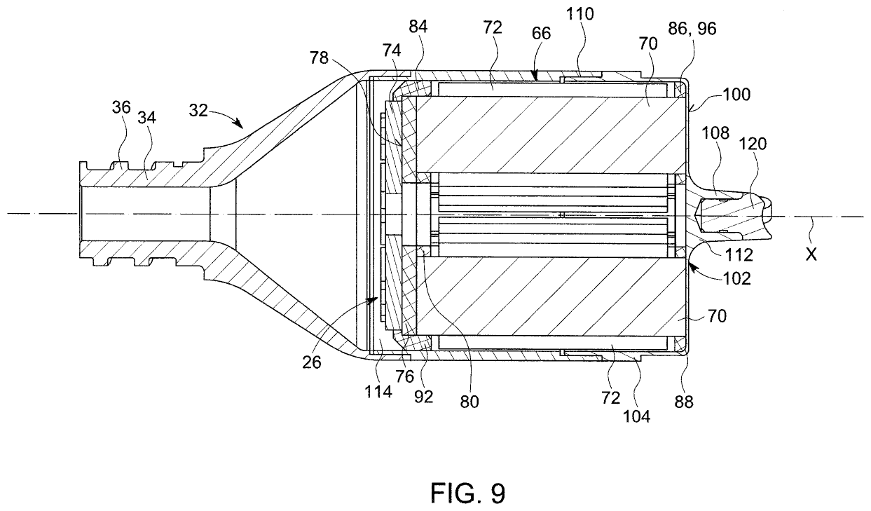

The drive unit casing 22 and the impeller casing 24 are made of titanium or a titanium alloy, which provides a high mechanical strength so that it allows to manufacture the drive unit casing 22 and the impeller casing 24 with a small thickness. Further, titanium has a good biocompatibility. The drive unit casing 22 and the impeller casing 24 are connected by e.g., gluing. The blood flow inlet 14 and the blood flow outlet 16 are both provided on the impeller casing 24. A drive unit 26 (see e.g., FIG. 9) is disposed within the drive unit casing 22. Particularities of the drive unit 26 will be explained below in more detail.

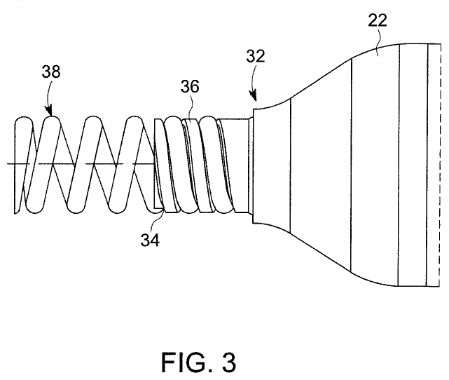

The impeller casing 24 comprises a cannula attachment portion 28 at one axial end opposite to the drive unit casing 22. The cannula attachment portion 28 is configured to receive a cannula (not shown) in a conventional manner. A catheter 30 is attached to a catheter attachment portion 32 of the drive unit casing 24. As shown in the detailed view of FIG. 3 with the catheter 30 removed, the catheter attachment portion 32 comprises a tubular portion 34 having a threading structure 36 on its outer peripheral surface which virtually corresponds to an external thread. The threading structure 36 is configured to threadedly engage a helical member in form of a Nitinol-coil 38 of the catheter 30 of the blood pump 10. In particular, the Nitinol-coil 38 of the catheter 30 is threaded onto the threading structure 36 in order to attach the catheter 30 to the pump housing 12.

### Impeller

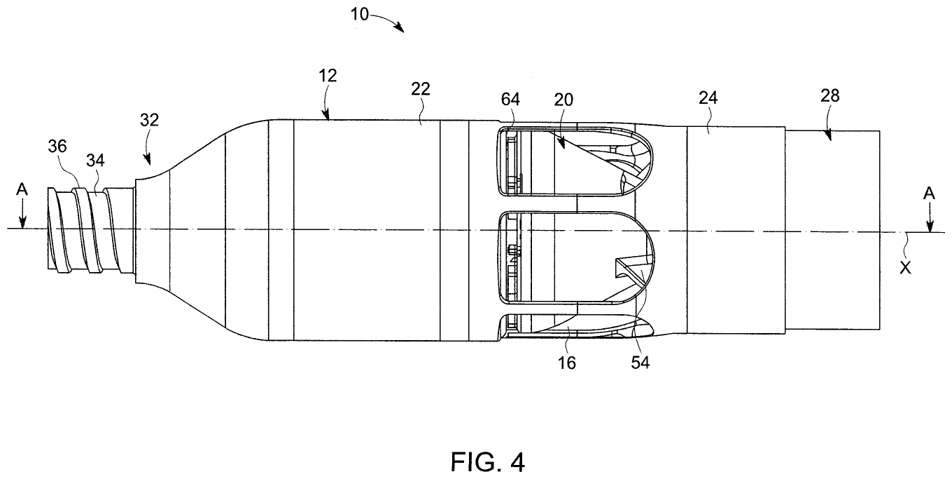

FIG. 5 illustrates a partial cross section along the line A-A shown in FIG. 4. Here, only the impeller casing 24 and the impeller 20 are shown in a cross-section. The impeller 20 is configured to convey blood along the passage 18 in that it is disposed within the impeller casing 24 and rotatable about a rotation axis X (see FIG. 4) by means of a bearing arrangement 40, 42. The rotation axis X coincides with the center axis of the pump housing 12. Here, the bearing arrangement 40, 42 comprises a first bearing 40 in form of a pivot bearing 40 and a second bearing 42 in form of a radial bearing 42. The impeller 20 comprises a main body 56 having a bearing accommodation portion 44 in its inside. The pivot bearing 40 is partially disposed in the bearing accommodation portion 44 as will be described in more detail below. The pivot bearing 40 allows for a certain amount of pivot movement of the impeller 20 relative to the pump housing 12.

The radial bearing 42 is supported at a crown 46 of the impeller casing 24. The crown 46 is provided adjacent to the blood flow inlet 14 and comprises a central tubular portion 48 connected to an inner peripheral surface of the impeller casing 24 by a plurality of connecting arms 50. In this embodiment, a total of three connecting arms 50 is provided, which are evenly distributed along the circumference of the central tubular portion 48 of the crown 46. Of course, it is also conceivable that only two or more than three connecting arms 50 are provided.

The impeller 20 further comprises a plurality of magnets 52 at one axial end, i.e. at the end pointing towards the drive unit casing 22. Rotation of the impeller 20 is caused by the drive unit 26 which is magnetically coupled to the impeller 20, as will be described in more detail below.

When the impeller 20 rotates about the axis of rotation X, blood is conveyed from the blood flow inlet 14 via the passage 18 to the blood flow outlet 16. Therefore, at least one primary blade 54 protrudes helically from an outer peripheral surface of the main body 56 of the impeller 20. In this embodiment, two primary blades 54 are provided. The primary blades 54 cause a primary blood flow along the passage 18.

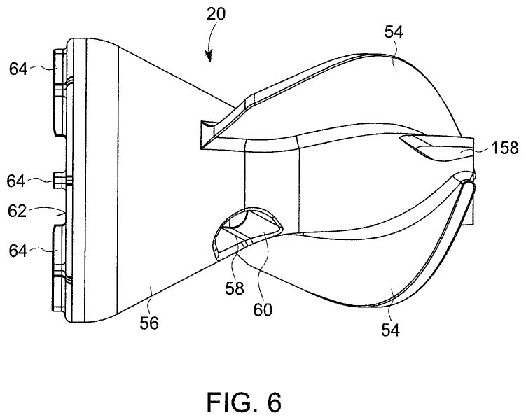

The impeller 20 further comprises at least one opening 58 connecting the passage 18 with the bearing accommodation portion 44. Here, two openings 58 are provided which both comprise an inlet 60 provided on the outer peripheral surface of the main body 56 of the impeller 20. As shown in FIGS. 5 and 6, the inlets 60 are provided circumferentially within the axial extension of the primary blades 54. In other words, at least a part of the primary blades 54 is provided adjacent to the inlets 60 in the circumferential direction of the main body 56 of the impeller 20. Each opening 58 has a center axis CA pointing towards the pivot bearing 40.

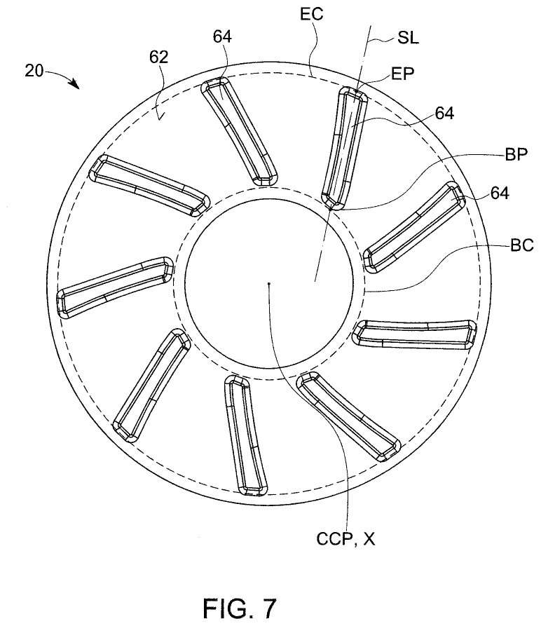

Further, the impeller has casing-side end 62 pointing towards the drive unit casing 22, see FIG. 7. A plurality of secondary blades 64 protrude from the casing-side end 62 in direction of the drive unit casing 22. The secondary blades 64 cause a secondary blood flow. The secondary blades 64 extend non-radially with respect to the axis of rotation X of the impeller 20.

Regarding the embodiment shown in FIG. 7, the secondary blades 64 each have a base point BP located on a base circle BC and an end point EP located on an end circle EC (in FIG. 7, the base point BP and the end point EP are only shown for one of the secondary blades 64). The base point BP is the radially innermost point of the secondary blade 64 and the end point EP is a radially outermost point of the secondary blade 64. The base circle BC and the end circle EC have a common center point CCP, through which the axis of rotation X runs. A straight line SL connecting the base point BP and the end point EP of each of the secondary blades 64 does not run through the common center point CCP. Thus, each of the plurality of secondary blades 64 is curved relative to the straight line SL.

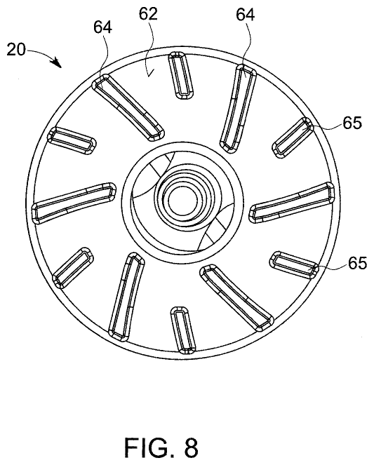

FIG. 8 shows an alternative embodiment comprising secondary main blades 64 and additional secondary auxiliary blades 65. The secondary auxiliary blades 65 are shorter in the radial direction compared to the secondary main blades 64 and are provided to be on the radial outer end circumference of the casing-side end 64 of the impeller 20.

### Drive Unit and Drive Unit Casing

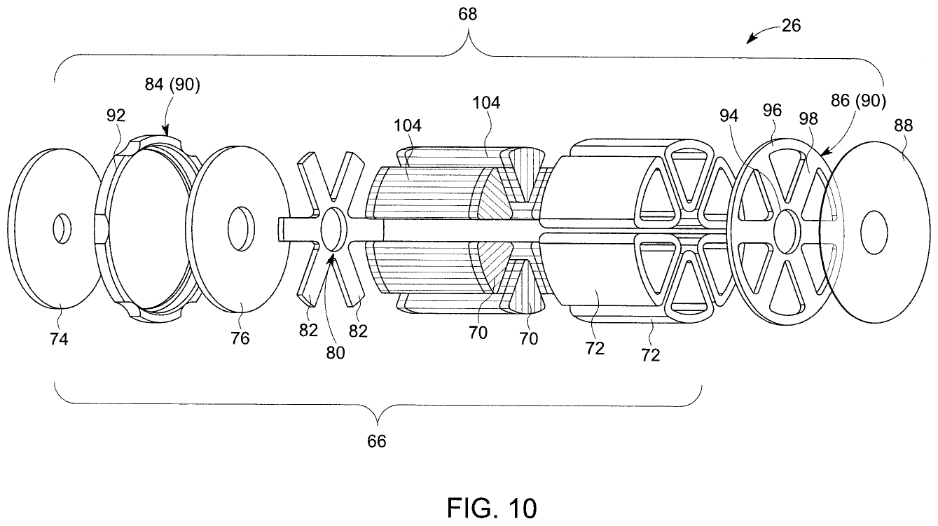

Referring now to FIGS. 9 and 10, the drive unit 26 will be explained in more detail. As mentioned above, the drive unit 26 is disposed within the drive unit casing 22, see e.g., FIG. 9. The drive unit 26 comprises a stator 66 and an insulation assembly 68. The drive unit 22 is configured to establish a rotating magnetic field which interacts with the magnets 52 of the impeller 20 to cause the impeller 20 to rotate about the axis of rotation X.

Therefore, the stator 66 comprises a plurality of posts 70 and a plurality of coil windings 72 disposed about the posts 70. The plurality of posts 70 are arranged in parallel to the axis of rotation X of the impeller 20. The coil windings 72 are sequentially controlled by a control element (e.g., a printed circuit board 74) to create the rotating magnetic field in a known manner. For enhancing the magnetic flux, the stator 66 further comprises a backplate 76 provided on a catheter-side end 78, i.e. that side of the stator 66 pointing to the catheter attachment portion 32 of the drive unit casing 22 in the assembled state.

In addition, the stator 66 comprises a backbone 80 having radially extending backbone legs 82. The backbone legs 82 space one of the plurality of posts 70 from an adjacent post, and hence, the number of backbone legs 82 equals the number of posts 70. The backbone legs 82 circumferentially space the posts 70 from each other. Here, the backbone 80 is star-shaped. In the assembled state of the stator 66, the backbone 80 is sandwiched between the backplate 76 and the coil windings 72. The plurality of posts 70, the backplate 76 and the backbone 80 are composed of a soft magnetic material, such as electrical steel or a suitable alloy, preferably cobalt steel. Preferably, the plurality of posts 70, the backplate 76 and the backbone 80 are composed of the identical material. In the embodiment shown, there are six posts 70 provided, but the number of posts 70 is of course not limited thereto.

The insulation assembly 68 comprises a spacer 84, a frontplate 86 and a frontsheet 88. The spacer 84 and the frontplate 86 may be considered to constitute a spacer assembly 90. The spacer 84 has a tubular portion 92 extending circumferentially and in the axial direction. In the assembled state of the drive unit 26, the spacer 84 is provided on the catheter-side end 78 of the stator 66 so that the backplate 76 and the backbone 82 are located radially inwardly of the spacer 84. In particular, the outer circumferential surface of the backplate 76 and the outer peripheral surface of each of the backbone legs 82 contact the inner peripheral surface of the tubular portion 92 of the spacer 84. The outer peripheral surface of the tubular portion 92 abuts against an inner surface of the drive unit casing 22, in particular against an inner peripheral surface of the drive unit casing 22. The diameter of spacer 84 is larger than the diameter of the stator 66. Further, the printed circuit board 74 is partially accommodated by the spacer 82, as shown in FIG. 9

The frontplate 86 comprises a central portion 94 and a ring-shaped outer portion 96 The central portion 94 and the outer portion 96 are connected by frontplate legs 98. The number of frontplate legs 98 equals the number of posts 70. The frontplate legs 98 circumferentially space the posts 70 from each other, in that the frontplate 86 is provided at an impeller-side end 100 of the stator 66, i.e. the end facing the impeller 20 in the assembled state of the drive unit 26. The frontplate 86 has a diameter identical to that of the spacer 84, so that the outer peripheral surface of the outer portion 96 contacts the inner peripheral surface of the drive unit casing 22. Hence, the spacer 82 and the frontplate 96 radially space the stator 66 from the inner peripheral surface of the drive unit casing 22, so that there is no contact between any portion or member of the stator 66 and the drive unit casing 22 in the radial direction.

Further, the frontsheet 88 covers the impeller-side end 100 of the stator 66 so as to inhibit contact between the stator 66 and the drive unit casing 22 in the axial direction. In particular, the frontsheet 88 inhibits contact between stator 66 and an impeller supporting portion 102 of the drive unit casing 22. The frontsheet 88 is a foil-like member having a thickness of about 3 to 9 μm, preferably of about 6 μm.

The spacer 82, the frontplate 86 and the frontsheet 88 are made of a non-electrical conductive material which is also a non-magnetizable material. Preferably the spacer 82, the frontplate 86 and the frontsheet 88 are made of a thermoplastic material, like polyetheretherketone (PEEK).

In addition, the insulation assembly 68 comprises a plurality of shrink elements 104. In particular, each of the plurality of posts 70 is surrounded circumferentially by one of the shrink elements 104 so as to inhibit direct contact between a post 70 and a coil winding 72 surrounding the respective post 70. In other words, the shrink elements 104 electrically separate the respective post 70 from the respective coil winding 72. As a shown in FIG. 10, it is not necessary that a shrink elements 104 extend along the entire axial extension of a post 70. Rather, it suffices when a shrink element 104 extends along about 50% or more of the axial extension of the respective post 70. In this embodiment, the shrink elements 104 are heat shrink sleeves composed of polyester.

In essence, the insulation assembly 68 greatly inhibits electrical leakage in that contact between the stator 66 and any part of the drive unit casing 22 is avoided. In addition, in adapting the dimensions of the different members of the insulation assembly 68, further properties can be adjusted. In addition, it is also possible to provide the frontplate 86 and the frontsheet 90 as a one-piece unitary member.

To further enhance the capability of reducing electrical leakage, the inner surface of the drive unit casing 22 may partially or completely be coated with a suitable coating, such as diamond-like carbon (DLC). In addition, a further shrink element may be provided surrounding the entire stator 66 circumferentially. Additionally or alternatively, the stator 66 may be circumferentially coated by suitable coating, such as diamond-like carbon (DLC). In addition, the plurality of posts 70 and the backplate 76 and the backbone 80 may be partially or entirely coated with a non-conductive material, in particular with DLC.

The impeller supporting portion 102 comprises a tubular member 104, a film-like portion 106 and a protruding pin 108. The impeller supporting portion is one part of the drive unit casing 22 and is connected to the other part via e.g., gluing or press fitting. In particular, the tubular member 104 is connected to a connecting portion 110 of another part of the drive unit casing 22.

The film-like portion 106 may contact the frontsheet 88 of the insulation assembly 68. The film-like portion 106 has a thickness of only about 60 to 80 μm, preferably of 70 μm. The protruding pin 108 protrudes from the film-like portion 106 in a direction towards the impeller 20. The main axis of the protruding pin 108 is concentric with the axis of rotation X. In this embodiment, the protruding pin 108 is integrally formed with the film-like portion 106 in that a rounded and smooth transition portion 112 is formed between the film-like portion 106 and the protruding pin 108 to reduce mechanical stress during rotation of the impeller 20. The protruding pin 108 further supports a part of the pivot bearing 42, namely a second pivot bearing member 120, as will be described in more detail below. The second pivot bearing member 120 may be glued or press fitted to the protruding pin 108.

As the film-like portion 106 has a relatively small thickness the mechanical stability of the film-like portion 106 is not as high as the mechanical stability of the tubular member 104 of the impeller supporting portion 102. To account therefore, the drive unit casing 22 is at least partially filed with a potting material 114. In particular, the potting material 114 may cover the stator 66 and the insulation assembly 68 and may thus fill the drive unit casing 22 at least between the impeller supporting portion 102 and the printed circuit board 74. The potting material 114 stiffens the film-like portion 106 from the inside of the drive unit casing 22 so as to reduce the risk of stress cracking or the like.

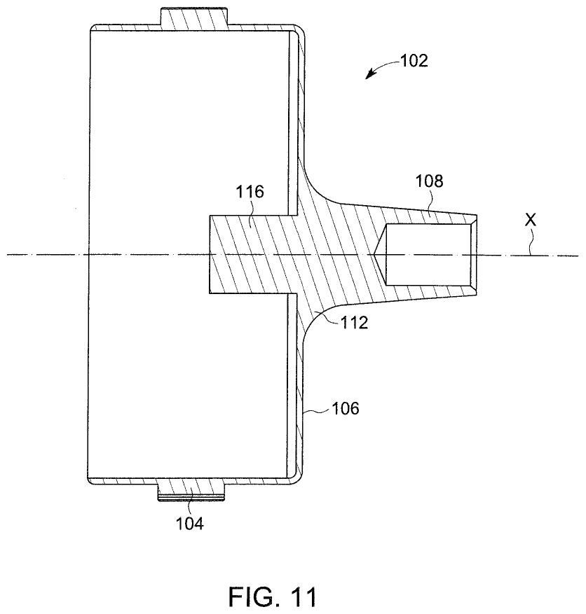

To further stiffen the impeller supporting portion 102, a stiffening member 116 may be provided, as shown in FIG. 11, which is an alternative embodiment of an impeller supporting portion 102. The stiffening member 116 protrudes from the film-like portion 106 of the impeller supporting portion 102 in a direction towards the stator 66. In this embodiment, the stiffening member 116 is a pin-like member integrally formed with film-like portion 106. In the assembled state of the blood pump 10, the stiffening member 116 protrudes into the stator 66 and is surrounded by the potting material 114. Thus, the potting material 114 further stiffens and reinforces the film-like portion 106.

In this embodiment, the potting material 114 is preferably a material having an FDA certification. Preferably, the potting material is a mixture of an epoxy resin and a metal oxide, for example aluminum oxide. For instance, a mixture of EpoTek® 301 and Al₂O₃ powder can be used. Preferably, EpoTek® 301 and Al₂O₃ powder in a ratio of 1:1.5 is used.

### Bearing Arrangement

Next, the bearing arrangement 40, 42 will be described in more detail.

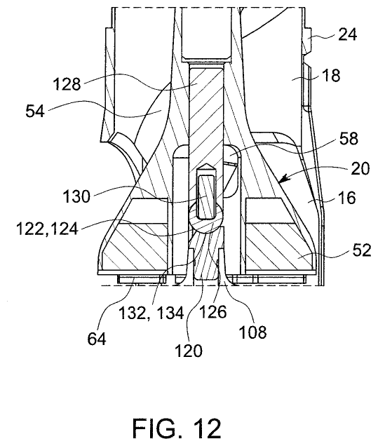

First, referring to FIG. 12 the first bearing 40 in form of the pivot bearing 40 is explained. The pivot bearing 40 comprises a first bearing member being a first pivot bearing member 118 and a second bearing member being the second pivot bearing member 120. The first pivot bearing member 118 is attached to the impeller 20. In particular, the first pivot bearing member 118 is disposed within the bearing accommodation portion 44 of the impeller 20. The second pivot bearing member 120 is accommodated at an axial end of the protruding pin 108. The first pivot bearing member 118 may be glued or press fitted to the bearing accommodation portion 44. The second pivot bearing member 120 may be glued or press fitted to the protruding pin 108.

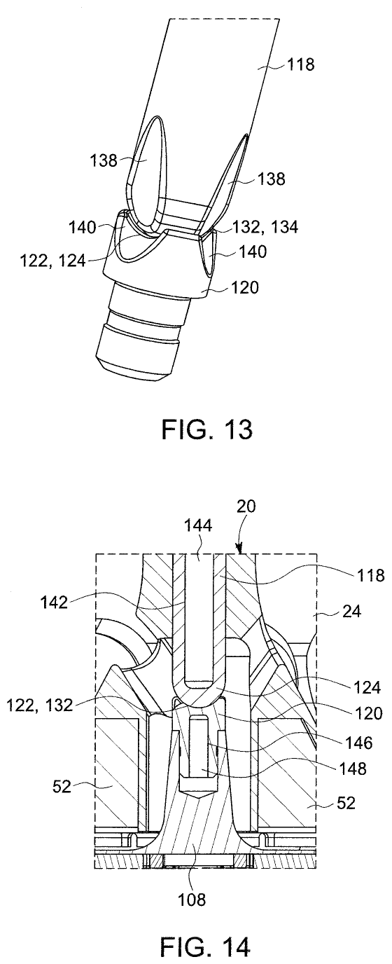

The first pivot bearing member 118 comprises a first abutment portion 122 having a spherical portion 124 with a convex surface. In the embodiment shown in FIG. 12, the spherical portion 124 is a part of a ball 126 connected to a pin-like support element 128 of the first pivot bearing member 118. In this embodiment, the ball 126 is fixed to the support element 128 by means of a fitting pin 130, but the spherical portion 122 can also be integrally formed with the support element 128, as shown in e.g., FIG. 13 or 14.

The second pivot bearing member 120 comprises a second abutment portion 132 having a first spherical cap 134 with a concave surface. The first spherical cap 134 may be a first calotte. The first abutment portion 122 abuts the second abutment portion 132 in that the spherical portion 124 contacts the first spherical cap 134. The contact between the spherical portion 124 and the first spherical cap 134 may be a point contact because the spherical portion 124 may have a first radius which is smaller than a second radius of the first spherical cap 134. A point contact can reduce the overall wear and mechanical stress during rotation of the impeller 20.

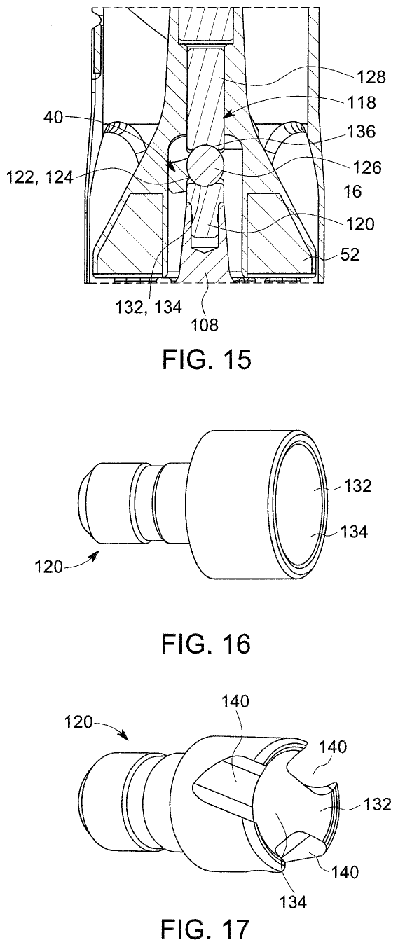

In an alternative embodiment shown in FIG. 15, the ball 126 is not fixed to the support element 128, but rather is supported in a second spherical cap 136 provided at an axial end of the support element 128. The second spherical cap 136 also comprises a concave surface with a third radius, which is larger than the first radius of the spherical portion 124 or the ball 126 respectively. Thus, the contact between the ball 126 and the second spherical cap 136 may also be point contact, which again allows to reduce the mechanical stress during rotation of the impeller 20.

As shown in an alternative embodiment according to FIG. 13, the first abutment portion 122 may comprise a plurality of first cut outs 138 distributed evenly along the circumference of the first abutment portion 122. As shown, the first cut outs 138 have an axial extension which is parallel to the axial extension of the support element 128. Further, each of the first cut outs 138 tapers from the first abutment portion 122 towards the other axial end of the support element 128. In particular, the first cut outs 138 are slanted towards a central axis of support element 128 which is concentric with the axis of rotation X of the impeller 20.

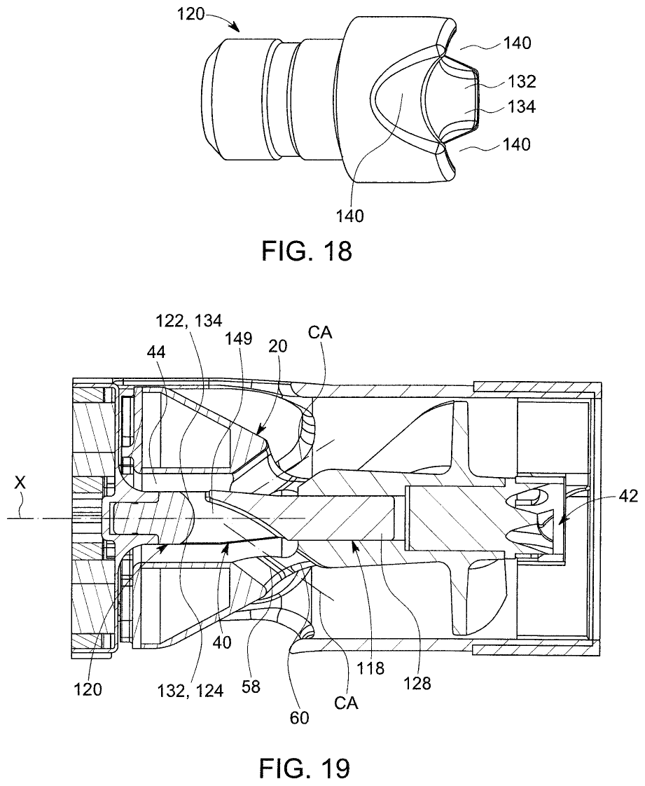

In FIGS. 16 to 18 alternative embodiments of the second pivot bearing member 120 are shown. The second abutment portions 132 of the embodiments shown in FIGS. 17 and 18 can comprise second cut outs 140, whereas the abutment portion 132 of the embodiment shown in FIG. 16 does not comprise any cut outs. In the embodiment according to FIG. 17, the second cut outs 140 are distributed evenly along the circumference of the second abutment portion 132. The axial extension of the second cut outs 140 is non-parallel with respect to a central axis of the second pivot bearing member 120, whereas the latter is concentric with the axis of rotation X of the impeller 20.

In the alternative embodiment shown in FIG. 18, the second cut outs 140 have an axial extension parallel to the central axis of the second pivot bearing member 120. In addition, the second cut outs 140 of the embodiment shown in FIG. 18 taper along their axial extension and may be slanted relative to the central axis of the second pivot bearing member 120.

In the embodiments shown, a total number of three first cut outs 138 and three second cut outs 140 is provided. However, the number of cut outs may be different between the first abutment portion 122 and the second abutment portion 132. In addition, more or less than three first cut outs 138 and second cut outs 140 may be provided. Further, it is also possible that only one of the first abutment portion 122 and the second abutment portion 132 comprises cut outs.

The first cut outs 138 and the second cut outs 140 are intended to facilitate blood flow when the impeller 20 rotates, so that the pivot bearing 40 may be cooled. In addition, the first cut outs 138 and the second cut outs 140 ameliorate the rinsing capability of the pivot bearing 40 and thus avoid blood particles to accumulate, i.e. blood clotting.

As mentioned above, the impeller 20 comprises openings 60 having a center axis CA pointing towards the pivot bearing 40. In particular, the center axis CA of the openings 60 points towards the first abutment portion 122 or the second abutment portion 132 respectively. Thus, the blood following through the openings 60 is directed towards the area of contact between the first abutment portion 122 and the second abutment portion 132 so as to cool and rinse the area of contact between the first abutment portion 122 and the second abutment portion 132.

To further enhance the cooling, it is possible to provide a first hollow portion 142 in the first pivot bearing member 118, see FIG. 14. The first hollow portion 142 is filed with a material having a higher thermal conductivity than the material of the of the first pivot bearing member 118. In the embodiment shown, a pin-like first cooling member 144 is disposed in the first hollow portion 142. The first cooling member 144 takes up some of the heat generated in the first abutment portion 122 when the impeller 20 rotates and distributes the same along the axial extension of the impeller 20.

The second pivot bearing member 120 may comprise a second hollow portion 146. The second hollow portion 146 is filed with a material having a higher thermal conductivity than the material of the of the second pivot bearing member 120. In the embodiment shown, a pin-like second cooling member 148 is disposed in the second hollow portion 146. The second cooling member 148 takes up some of the heat generated in the second abutment portion 132 when the impeller 20 rotates and distributes the same along the axial extension of the protruding pin 108.

The first pivot bearing member 118 may entirely or partly be composed of a first ceramic material chosen from silicon carbide (SIC), aluminum toughened zirconia (ATZ), zirconia toughened aluminum (ZTA) or aluminum oxide (Al₂O₃). Alternatively, the first pivot bearing member 118 may entirely of partly be composed of a metallic material, like cemented carbide. The metallic material may further be coated with the first ceramic material. The metallic material or the first ceramic material may further be coated with DLC. The DLC coating may comprise a boron-doped DLC film. The boron-doped DLC film may be deployed on a non-boron-doped DLC interlayer to improve adhesion. Alternatively, the first pivot bearing member 118 may entirely or partly be composed of diamond.

The second pivot bearing member 120 may entirely of partly be composed of a second ceramic material chosen from SIC, ATZ, ZTA or Al₂O₃. Further, the second pivot bearing member 120 may entirely or partly be composed of a metallic material, such as cemented carbide. The metallic material may further be coated with the second ceramic material. The metallic material or the second ceramic material may further be coated with DLC. The DLC coating may comprise a boron-doped DLC film. The boron-doped DLC film may be deployed on a non-boron-doped DLC interlayer to improve adhesion. Alternatively, the second pivot bearing member 120 may entirely of partly be composed of diamond.

In case a ball 126 is provided, the ball may be partly or entirely composed of a third ceramic material chosen from SIC, ATZ, ZTA or Al₂O₃. Further, the ball 126 may entirely or partly be composed of a metallic material, such as cemented carbide. The metallic material may further be coated with the third ceramic material. The metallic material or the second ceramic material may further be coated with DLC. The DLC coating may comprise a boron-doped DLC film. The boron-doped DLC film may be deployed on a non-boron-doped DLC interlayer to improve adhesion. Alternatively, the ball 126 may entirely of partly be composed of diamond.

The first ceramic material, the second ceramic material and the third ceramic material may be different or identical. The following combinations of materials given in table 1 have been proven to be particularly suitable in terms of heat transfer, wear, friction, rinsing capability and avoidance of attachment of blood particles:

TABLE 1
Preferable material combinations

| First ceramic | Second ceramic | Third ceramic |
|---|---|---|
| material | material | material |
| SIC | SIC | N/A |
| Al₂O₃ | ATZ | N/A |
| ATZ | ZTA | N/A |
| ZTA coated with DLC | ATZ | N/A |
| ATZ | ATZ | ZTA |

Preferably, the first ceramic material of the first pivot bearing member 118 is ZTA and the second ceramic material of the second pivot bearing member 120 is ATZ.

As mentioned above, the first cooling member 144 and the second cooling member 148 are made of a material having a higher thermal conductivity than the material of the first pivot bearing member 118 and the second pivot bearing member 120 respectively. In particular, the first cooling member 144 and the second cooling member 148 are made of either silver, silver alloy, copper or copper alloy.

Of course, although it has been described above that the first pivot bearing member 118 comprises the spherical portion 124 and the second pivot bearing member 120 comprises the first spherical cap 134, the arrangement may be twisted in that the first pivot bearing member 118 comprises the first spherical cap and the second pivot bearing member 120 comprises the spherical portion. An according embodiment is shown in FIGS. 19 to 21 and will be described in the following.

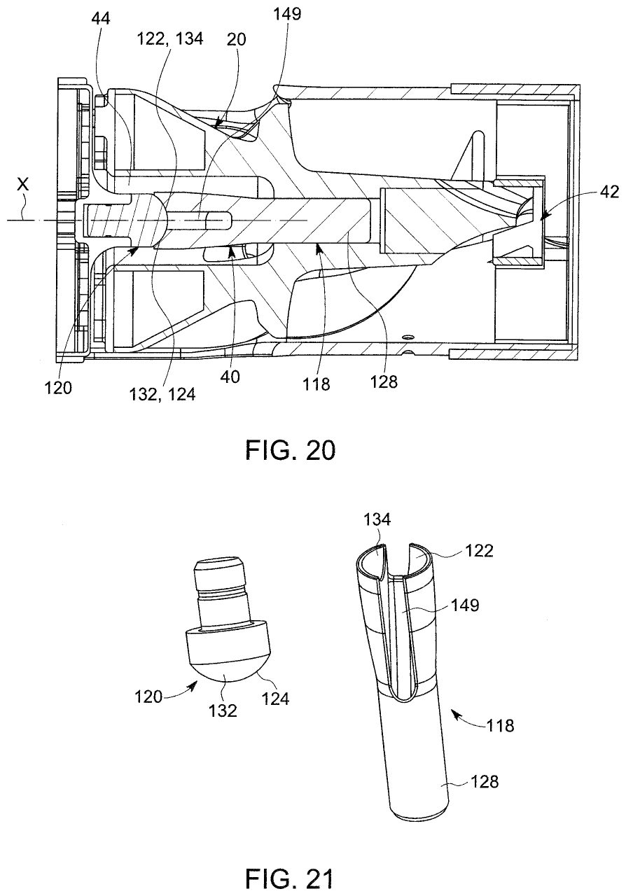

As illustrated in FIGS. 19 and 20, the first pivot bearing member 118 may comprise a first abutment portion 122 having a concave surface, i.e. a first spherical cap 134 or first calotte respectively. Accordingly, the second pivot bearing member 120 may comprise a second abutment portion 132 having a convex surface, i.e. a spherical portion 124.

The support element 128 comprises a slot 149, which extends from the first abutment portion 122. At an end of the support element 128 that faces the first abutment portion 122, the slot 149 extends over the entire diameter of the support element 128 and thus separates the first spherical cap 134 into two parts, see FIG. 21.

The slot 149 is configured such that a rotation of the first pivot bearing member 118 and/or of the support element 128 causes a pumping action from one lateral side of the slot 149 to another lateral side of the slot. The slot 149 is delimited by two parallel side surfaces. Preferably, the two side surfaces are parallel to each other and/or parallel to a middle plane of the support element 128. The slot 149 does not extend over the entire diameter of the support element 128 over the entire length of the slot 149. In a first section of the slot 149 facing the first abutment portion 122, the slot 149 extends over the entire diameter of the support element 128 (see FIGS. 19 and 21). Thus, the slot 149 extends to both lateral sides of the support element 128 in the first section of the slot 149. In a second section of the slot 149, the slot only extends to one lateral side of the support element 128. In particular, a slot width of the slot is gradually reduced in a direction away from the first abutment portion 122.

The support element 128 tapers in the axial direction from the first abutment portion 122 towards the radial bearing 42. In particular, the diameter of the support element 128 is larger along the axial extension of slot 149. Of course, the support element 128 may also have a cylindrical shape.

Further, the center axis CA of the opening 60 points towards the slot 149. During rotation of the impeller 20 blood enters the bearing accommodation portion 44 through the openings 60 and is guided within the slot 149 towards the second pivot bearing member 120. When exiting the slot 149 on a lateral side of the support element 128 in the contact area of the first abutment portion 122 and the second abutment portion 132 the contact area is cooled and rinsed.

Preferably, for the embodiment shown in FIGS. 19 to 21 the first ceramic material of the first pivot bearing member 118 is ATZ and the second ceramic material of the second pivot bearing member 120 is ZTA.

Further, the first abutment portion 122 may be coated with a diamond coating. In this regard, also the second abutment portion 132 may be coated with a diamond coating. The diamond coating may be a chemical vapor deposition (CVD) diamond coating. The diamond coating may be directly applied on a ceramic material as described above. Of course, the diamond coating may also be applied on a first abutment portion 122 or a second abutment portion 132 respectively made of another material e.g., titanium, titanium alloy or stainless steel.

Next, the radial bearing 42 will be described in more detail.

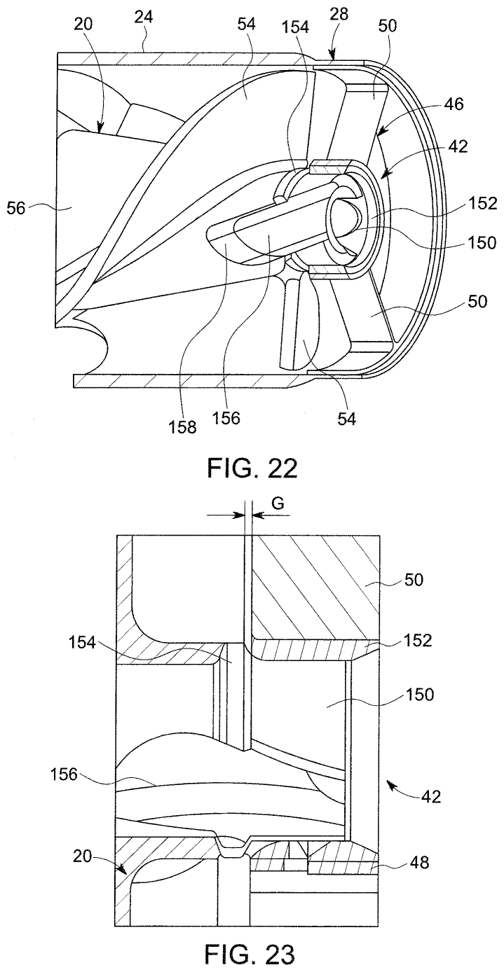

FIG. 22 shows the radial bearing 42 in detail in a perspective and partially cut view. The radial bearing 42 comprises a first radial bearing member 150 and a second radial bearing member 152. The first radial bearing member 150 is disposed at an axial end of the impeller 20 pointing towards the crown 46. The first radial bearing member 150 may be glued or press fitted to the impeller 20. The first radial bearing member 150 is roughly cylindrical and comprises a collar 154 in the form of a circumferential protrusion. In addition, the first radial bearing member 150 comprises third cut outs 156 which are evenly distributed circumferentially along an outer peripheral surface of the first radial bearing member 150.

As one can take from FIG. 22, the third cut outs 156 have an axial extension which is not parallel to the center axis of the first radial bearing member 150, the latter one being concentric with the axis of rotation X of the impeller 20. In the embodiment shown, a total of two third cut outs 156 are provided.

The outer peripheral surface of the main body 56 of the impeller 20 also comprises matching impeller cut outs 158 which smoothly prolong the third cut outs 156. The collar 154 abuts the main body 56 of the impeller 20 so as to limit the axial movement of the first radial bearing member 150 in an axial direction towards the stator 66.

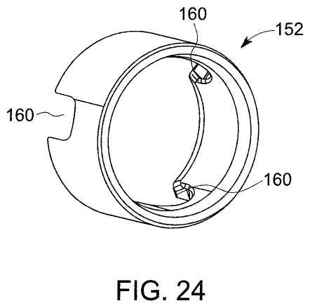

The second radial bearing member 152 is a ring shaped member disposed in the central tubular portion 48 of the crown 46, see FIGS. 22 and 23. The second radial bearing member 152 may be glued or press fitted to the central tubular portion 48 of the crown 46. As illustrated in FIG. 24, the second radial bearing member 152 comprises a plurality of fourth cut outs 160 provided evenly circumferential around the second radial bearing member 152. As shown, the fourth cut outs 160 extend radially through the second radial bearing member 152 and the open ends of the fourth cut outs 160 point towards the impeller 20. In this embodiment, three fourth cut outs 160 are provided. The circumferential extension of each of the fourth cut outs 160 is smaller than the circumferential distance between the third cut outs 156 of the first bearing member 150 to warrant that the first bearing member 150 is safely supported by the second bearing member 152 in the assembled state of the radial bearing 42.

The third cut outs 156, the impeller cut outs 158 and the fourth cut outs 160 are provided to cool the radial bearing by directing a part of the blood flow along the respective cut outs when the impeller 20 rotates. This further avoids accumulation of blood particles in the area of the radial bearing 42.

Further, the collar 146 does not contact the second radial bearing member 152 in the assembled state of the radial bearing 42. Rather, a gap G is formed between collar 154 and the second radial bearing member 152 in the axial direction during normal operating conditions of the blood pump 10, see FIG. 23. Even when the impeller 20 rotates to convey blood from the blood flow inlet 14 to the blood flow outlet 16 there is no contact between the collar 154 and the second radial bearing member 150 due to the attractive forces between the stator 66 and the magnets 52 of the impeller 20. In other words, the attractive forces act in direction of the stator 66 so that the impeller 20 does not move in the axial direction when rotating. However, the collar 154 forms an emergency stop to limit the axial movement of the impeller 20 in a direction away from the stator 66 in case of malfunction.

The first radial bearing member 150 and the second radial bearing member 152 may be composed of a ceramic material, a metallic material or diamond. The ceramic material may be SIC, ATZ, ZTA or Al₂O₃. The ceramic material of the first radial bearing member 150 and the second radial bearing member 152 may be identical or may be different. In addition, the ceramic material may be provided as a coating when the first radial bearing member 150 and/or the second radial bearing member 152 are made of a metallic material, in particular cemented carbide. Further, the metallic material or the ceramic material may be coated with DLC. The DLC coating may comprise a boron-doped DLC film. The boron-doped DLC film may be deployed on a non-boron-doped DLC interlayer to improve adhesion. The following material combination has been found to be particularly preferable: SIC and SIC, ATZ and Al₂O₃, ATZ and ZTA, and ATZ and ZTA coated with DLC.

Preferably, the ceramic material of the first radial bearing member 150 is ZTA and the ceramic material of the second radial bearing member 152 is ATZ.

Further, the first radial bearing member 150 may be coated with a diamond coating. In this regard, also the second radial bearing member 152 may be coated with a diamond coating. The diamond coating may be a chemical vapor deposition (CVD) diamond coating. The diamond coating may be directly applied on a ceramic material as described above. Of course, the diamond coating may also be applied on a first radial bearing member 150 or a second radial bearing member 152 respectively made of another material e.g., titanium, titanium alloy or stainless steel.

Generally, coating a ceramic material with DLC has the advantage that the emergency run properties of the bearing arrangement 40, 42 are relatively high, even in case the DLC coating has been damage or removed e.g., due to wear. Generally, a diamond coating has the advantages of high wear resistance and a high biocompatibility.

### Function of Blood Pump

A control comprising the printed circuit board 74 generates a rotating magnetic field within the stator 66 in a known manner which acts together with the magnets 52 of the impeller 20 so that the impeller 20 rotates about the axis of rotation X. Thereby, the primary blades 54 of the impeller 20 cause a primary blood flow from the blood flow inlet 14 via the passage 18 to the blood flow outlet 16. A part of the primary blood flow is directed along the radial bearing 42 along the third cut outs 156, the impeller cut outs 158 and the fourth cut outs 160 to cool and rinse the radial bearing 42 and to avoid accumulation of blood particles in the area of the radial bearing 42.

The secondary blades 64 of the impeller 20 cause a secondary blood flow and blood is dragged from the passage 18 through the openings 60 into the bearing accommodation portion 44. There, the secondary blood flow is guided either along first cut outs 138 and the second cut outs 140 (if provided) or the slot 149 to cool and rinse the pivot bearing 40 and to avoid accumulation of blood particles in the area of the pivot bearing 40. The secondary blood flow then exits the bearing accommodation portion 44 through the space formed between the impeller 20 and the impeller supporting portion 102 and exits the blood pump 10 through the blood flow outlet 16.

### EXEMPLARY IMPLEMENTATIONS

As already described, the technology described herein may be implemented in various ways. In that regard, the foregoing disclosure is intended to include, but not be limited to, the systems, methods, and combinations and subcombinations thereof that are set forth in the following exemplary implementations. Preferred embodiments are described in the following paragraphs:
A1 Blood pump, in particular intravascular blood pump, comprising: a pump housing having a blood flow inlet and a blood flow outlet connected by a passage, an impeller disposed in said pump housing, and a drive unit configured to drive the impeller.
A2 Blood pump according to paragraph A1, wherein the pump housing comprises a drive unit casing and wherein the drive unit is disposed within the drive unit casing, wherein the drive unit comprises a stator and an insulation assembly, and wherein the insulation assembly is configured to prevent electrical leakage.
A3 Blood pump according to paragraph A2, wherein the insulation assembly comprises a spacer assembly configured to space the stator from the drive unit casing so as to inhibit contact between the stator and the drive unit casing.
A4 Blood pump according to paragraph A3, wherein the spacer assembly comprises a spacer configured to radially space the stator from an inner surface of the drive unit casing, in particular an inner peripheral surface of the drive unit casing.
A5 Blood pump according to paragraph A4, wherein the stator has a catheter-side end pointing away from the impeller, wherein the spacer is ring-shaped and has tubular portion, the tubular portion extending at least partially circumferentially, and wherein the spacer is disposed at the catheter-side end of the stator so that the stator is partially disposed radially inside of the tubular portion of the spacer.
A6 Blood pump according to paragraph A5, wherein the tubular portion of the spacer abuts against the inner surface of the drive unit casing.
A7 Blood pump according to paragraph A5 or A6, wherein the stator comprises a backplate contacting the catheter-side end of the stator, wherein the backplate is disposed radially inside of the tubular portion of the spacer.
A8 Blood pump according to any one of the preceding paragraphs A3 to A7, wherein the drive unit further comprises a printed circuit board, wherein the printed circuit board is at least partially disposed radially inside of the spacer assembly.
A9 Blood pump according to any one of the preceding paragraphs A2 to A8, wherein the insulation assembly comprises a frontplate configured to space the stator from the drive unit casing so as to inhibit contact between the stator and the drive unit casing.
A10 Blood pump according to paragraph A9, wherein the stator comprises a plurality of posts, and coil windings disposed about the posts, wherein the frontplate has a central portion and an outer portion, wherein a plurality of frontplate legs extend between the central portion and the ring-shaped outer portion.
A11 Blood pump according to paragraph A10, wherein the frontplate legs are configured to circumferentially space each of the plurality of posts from an adjacent post, and wherein the plurality of posts are partially disposed radially inwardly of the ring-shaped outer portion, wherein the outer portion is preferably ring-shaped.
A12 Blood pump according to any one of the preceding paragraphs A1 to A11, wherein the stator comprises a plurality of posts, and coil windings disposed about the posts.
A13 Blood pump according to any one of the preceding paragraphs A10 to A12, wherein the stator comprises a backbone having radially extending backbone legs, wherein the backbone legs are preferably configured to circumferentially space each of the plurality of posts from an adjacent post.
A14 Blood pump according to any one of the preceding paragraphs A10 to A13, wherein the insulation assembly comprises a plurality of shrink elements wherein each of the plurality of posts is at least partially surrounded by one of the shrink elements.
A15 Blood pump according to any one of the preceding paragraphs A2 to A14, wherein the stator has an impeller-side end pointing towards the impeller, wherein the insulation assembly further comprises a frontsheet, and wherein the frontsheet covers the impeller-side end of the stator so as to inhibit contact between the stator and the drive unit casing.
A16 Blood pump according to any one of the preceding paragraphs A2 to A15, wherein the insulation assembly is at least partially composed of a non-electrical conductive material, wherein the insulation assembly is preferably entirely composed of the non-electrical conductive material.
A17 Blood pump according to claim A16, wherein the non-electrical conductive material is a thermoplastic material, preferably a polyaryletherketone, wherein the non-electrical conductive material is preferably polyetheretherketone.
A18 Blood pump according to any one of the preceding paragraphs A1 to A17, wherein the drive unit casing is made of titanium or titanium alloy.
A19 Blood pump according to any one of the preceding paragraphs A1 to A18, wherein an inner surface of the drive unit casing is at least partially coated with a non-electrical conductive coating, in particular diamond-like carbon (DLC).
A20 Blood pump according to any one of the preceding paragraphs A1 to A19, wherein the stator is at least partially surrounded by a shrink element circumferentially and/or the stator is at least partially coated with a non-electrical conductive coating.
A21 Blood pump according to any one of the preceding paragraphs A1 to A20, wherein the blood pump further comprises a bearing arrangement rotatably supporting the impeller, wherein the bearing arrangement comprises at least one bearing comprising a first bearing member and a second bearing member, wherein the first bearing member comprises a first abutment portion and wherein the second bearing member comprises a second abutment portion, wherein the first abutment portion comprises a first ceramic material or is composed of a first ceramic material and/or wherein the second abutment portion comprises a second ceramic material or is composed of a second ceramic material.
A22 Blood pump according to paragraph A21, wherein the first ceramic material is different from the second ceramic material or wherein the first ceramic material is identical to the second ceramic material.
A23 Blood pump according to paragraph A21 or A22, wherein the first bearing member is entirely composed of the first ceramic material and/or wherein the second bearing member is entirely composed of the second ceramic material.
A24 Blood pump according to paragraph A21 or A22, wherein the first abutment portion is composed of a metallic material, in particular cemented carbide, coated with the first ceramic material and/or wherein the second abutment portion is composed of a metallic material, in particular cemented carbide, coated with the second ceramic material.
A25 Blood pump according to any one of the preceding paragraphs A21 to A24, wherein the first abutment portion is coated with diamond-like carbon (DLC) and/or wherein the second abutment portion is coated with diamond-like carbon (DLC).
A26 Blood pump according to any one of the preceding paragraphs A21 to A25, wherein the first ceramic material is chosen from SIC, ATZ, ZTA or Al₂O₃.
A27 Blood pump according to any one of the preceding paragraphs A21 to A26, wherein the second ceramic material is chosen from SIC, ATZ, ZTA or Al₂O₃.
A28 Blood pump according to any one of the preceding paragraphs A21 to A27, wherein the first bearing member is at least partially composed of diamond or wherein the second bearing member is at least partially composed of diamond.
A29 Blood pump according to any one of the preceding paragraphs A1 to A19, wherein the blood pump further comprises a bearing arrangement rotatably supporting the impeller, wherein the bearing arrangement comprises at least a first bearing comprising a first bearing member and second bearing member, the first bearing member comprising a first abutment portion and the second bearing member comprising a second abutment portion, and wherein the first abutment portion is coated with DLC and/or wherein the second abutment portion is coated with DLC.
A30 Blood pump according to any one of the preceding paragraphs A25 to A29, wherein the DLC coating comprise or consists of a boron-doped DLC film.
A31 Blood pump according to any one of the preceding paragraphs A30, wherein the DLC coating comprises a DLC interlayer, wherein the boron-doped DLC film is deployed on the DLC interlayer.
A32 Blood pump according to paragraph A30 or A31, wherein the boron-doped DLC film comprises a boron to carbon ratio of at 0.01 to 0.4. preferably of 0.03 to 0.1, and most preferably of 0.03.
A33 Blood pump according to any one of the preceding paragraphs A1 to A19, wherein the blood pump further comprises a bearing arrangement rotatably supporting the impeller, wherein the bearing arrangement comprises at least a first bearing comprising a first bearing member and second bearing member, the first bearing member comprising a first abutment portion and the second bearing member comprising a second abutment portion, and wherein the first abutment portion is coated with a diamond coating and/or wherein the second abutment portion is coated with a diamond coating.
A34 Blood pump according to paragraph A33, wherein the diamond coating is a a chemical vapor deposition (CVD) diamond coating or a high-pressure high-temperature (HPHT) diamond coating.
A35 Blood pump according to any one of the preceding paragraphs A21 to A34, wherein the first abutment portion at least partially contacts the second abutment portion.
A36 Blood pump according to paragraph A35, wherein the contact between the first abutment portion and the second abutment portion is a point contact.
A37 Blood pump according to any one of the preceding paragraphs A21 to A36, wherein the first bearing member comprises a support element and a ball, wherein the support element comprises the first abutment portion, and wherein the ball abuts the first abutment portion and the second abutment portion.
A38 Blood pump according to paragraph A37, wherein the ball comprises a third ceramic material or is composed of a third ceramic material and the third ceramic material is chosen from SIC, ATZ, ZTA or Al₂O₃.
A39 Blood pump according to paragraph A37, wherein the ball comprises a metallic material, in particular cemented carbide, or is composed of metallic material, in particular cemented carbide.
A40 Blood pump according to any one the preceding paragraphs A37 to A39, wherein the ball is coated with diamond-like carbon (DLC).
A41 Blood pump according to paragraph A37, wherein the ball comprises diamond or is composed of diamond.
A42 Blood pump according to any one of the preceding paragraphs A21 to A41, wherein the first bearing member comprises a first hollow portion filed a with a material having a higher thermal conductivity than a material of the first bearing member, and/or wherein the second bearing member comprises a second hollow portion filed with a material having a higher thermal conductivity than a material of the second bearing member, wherein the material with high thermal conductivity preferably is one of silver, silver alloy, copper, copper alloy and diamond.
A43 Blood pump according to any one of the preceding paragraphs A21 to A42, wherein the bearing arrangement comprises a first bearing and a second bearing.
A44 Blood pump according to any paragraph A43, wherein the first bearing is a pivot bearing and wherein the second bearing is a radial bearing.
A45 Blood pump according to any one of the preceding paragraphs A1 to A44, wherein the blood pump further comprises a bearing arrangement rotatably supporting the impeller, wherein the bearing arrangement comprises at least one pivot bearing.
A46 Blood pump according to paragraph A45, wherein the pivot bearing comprises a first pivot bearing member and a second pivot bearing member, wherein the first pivot bearing member is pivotable relative to the second pivot bearing member, wherein the first pivot bearing member comprises a first abutment portion and wherein the second pivot bearing member comprises a second abutment portion, and wherein the first abutment portion of the first pivot bearing member at least partially contacts the second abutment portion of the second pivot bearing member.
A47 Blood pump according to paragraph A46, wherein the contact between the first abutment portion of the first pivot bearing member and the second abutment portion of the second pivot bearing member is a point contact.
A48 Blood pump according to paragraph A46 or A47, wherein the first abutment portion of the first pivot bearing member comprises a spherical portion and wherein the second abutment portion of the second pivot bearing member comprises a first spherical cap, wherein the spherical portion abuts the spherical cap.
A49 Blood pump according to paragraph A48, wherein the first spherical cap is a first calotte.
A50 Blood pump according to paragraph A48 or A49, wherein the first spherical cap has a concave surface and/or wherein the spherical portion has a convex surface.
A50 Blood pump according to any one of the preceding paragraphs A48 to A50, wherein the spherical portion has a first radius and the first spherical cap has a second radius, wherein the second radius is larger than the first radius.
A52 Blood pump according to any one of the preceding paragraphs A48 to A50, wherein the first pivot bearing member comprises a support element and a ball, wherein the ball abuts the support element and comprises the spherical portion.
A53 Blood pump according to paragraph A52, wherein the support element comprises a second spherical cap with a third radius, wherein the ball abuts the second spherical cap and wherein the third radius is larger than the first radius.
A54 Blood pump according to paragraph A53, wherein the second spherical cap is a second calotte.
A55 Blood pump according to paragraph A53 or A54, wherein the second spherical cap has a concave surface.
A56 Blood pump according to any one of the preceding paragraphs A52 to A55, wherein the ball is fixed to the support element.
A57 Blood pump according to any one of the preceding paragraphs A46 to A56, wherein the first abutment portion of the first pivot bearing member comprises at least one first cut out, preferably a plurality of first cut outs distributed evenly along the circumference of the first abutment portion.
A58 Blood pump according to claim any one of the preceding paragraphs A46 to A57, wherein the second abutment portion of the second pivot bearing member comprises at least one second cut out, preferably a plurality of second cut outs distributed evenly along the circumference of the second abutment portion.
A59 Blood pump according to paragraph A58, wherein the axial extension of the at least one second cut out is non-parallel to a main axis of the second pivot bearing member or wherein the axial extension of the at least one second cut out is parallel to the main axis of the second pivot bearing member.
A60 Blood pump according to paragraph A46, wherein the first pivot bearing member comprises a slot extending from the first abutment portion.
A61 Blood pump according to paragraph A60, wherein the slot separates the first abutment portion of the first pivot bearing member into two parts.
A62 Blood pump according to paragraph A60 or A61, wherein the slot is configured such that a rotation of the first pivot bearing member causes a pumping action from one lateral side of the slot to another lateral side of the slot.
A63 Blood pump according to any one of the preceding paragraphs A60 to A62, wherein
   a) the slot is delimited by two parallel side surfaces, wherein the two side surfaces are preferably parallel to each other and/or parallel to a middle plane of the support element and/or wherein
   b) the slot does not extend over the entire diameter of the support element over the entire length of the slot.
A64 Blood pump according to any one of the preceding paragraphs A46 to A63, wherein the impeller comprises a bearing accommodation portion in its inside and at least one opening connecting the passage with the bearing accommodation portion, wherein the pivot bearing is at least partially disposed within the bearing accommodation portion.
A65 Blood pump according to paragraph A64, wherein the opening has a center axis pointing towards the second abutment portion of the second pivot bearing member.
A66 Blood pump according to paragraph A64 or A65, wherein the impeller comprises a main body and at least one primary blade protruding helically from an outer peripheral surface of the main body, wherein the opening comprises an inlet provided on the outer peripheral surface of the main body, and wherein at least a part of the at least one primary blade is disposed next to the inlet in the circumferential direction of the main body.
A67 Blood pump according to paragraph A66, wherein the inlet is disposed circumferentially within an axial extension of the at least one primary blade.
A68 Blood pump according to any one of the preceding paragraphs A46 to A67, wherein the first pivot bearing member is attached to the impeller and wherein the second pivot bearing member is attached to the pump housing.
A69 Blood pump according to any one of the preceding paragraphs A45 to A68, wherein the bearing arrangement further comprises at least one radial bearing, wherein the pivot bearing rotatably supports the impeller relative to the housing at one point of the impeller and wherein the radial bearing rotatably supports the impeller relative to the housing at another point of the impeller, wherein the drive unit is an non-contact electromagnetic drive unit configured to rotatably drive the impeller relative to the housing, and wherein the drive unit further establishes an attractive force on the impeller
A70 Blood pump according to paragraph A69, wherein the attractive force is established in a direction away from the blood flow inlet, so that no axial force acts on the radial bearing.
A71 Blood pump according to paragraph A59 or A70, wherein the radial bearing comprises a first radial bearing member and a second radial bearing member, wherein the first radial bearing member is attached to the impeller and wherein the second radial bearing member is attached to the pump housing, wherein the first radial bearing member comprises a collar, wherein the collar extends at least partially circumferentially from an outer peripheral surface of the first radial bearing member, wherein the collar is provided adjacent the second radial bearing member, and wherein a gap is formed between the collar and the second radial bearing member in the axial direction.
A72 Blood pump according to paragraph A71, wherein no axial forces act on the collar.
A73 Blood pump according to paragraph A71 or A72, wherein the collar is an emergency abutment collar limiting axial movement of the impeller relative to the housing.
A74 Blood pump according to any one of the preceding paragraphs A45 to A73, wherein the pivot bearing members are attached via gluing and/or press-fitting and/or wherein the radial bearing members are attached via gluing and/or press fitting.
A75 Blood pump according to any one of the preceding paragraphs A1 to A74, wherein the blood pump further comprises a stator, wherein the pump housing comprises a drive unit casing with a catheter attachment portion at one axial end and an impeller supporting portion at the other axial end, wherein the stator is disposed within the drive unit casing, and wherein the drive unit casing is at least partially filled with a potting material.
A76 Blood pump according to paragraph A75, wherein the potting material contacts the impeller supporting portion.
A77 Blood pump according to paragraph A76, wherein the potting material comprises an epoxy resin, preferably a mixture of an epoxy resin and aluminum oxide, preferably a mixture of EpoTek® 301 and Al₂O₃ powder, preferably in a ratio of 1:1.5.
A78 Blood pump according to any one of the preceding paragraphs A75 to A77, wherein the impeller supporting portion comprises a film-like portion and a protruding pin, wherein the protruding pin is configured to rotatably support the impeller.
A79 Blood pump according to paragraph A78, wherein the film-like portion has a thickness of 60 μm to 80 μm, preferably of 70 μm.
A80 Blood pump according to paragraph A78 or A79, wherein the protruding pin is integrally formed with the film-like portion.
A81 Blood pump according to any one of the preceding paragraphs A78 to A80, wherein the center axis of the protruding pin is concentric with the axis of rotation of the impeller.
A82 Blood pump according to any one of the preceding paragraphs A78 to A81, wherein a rounded transition portion is provided between the film-like portion and the protruding pin.
A83 Blood pump according to any one of the preceding paragraphs A75 to A82, wherein at least one stiffening member protrudes from the impeller supporting portion in a direction of the catheter attachment portion, wherein the stiffening member preferably protrudes into an interior of the drive unit casing.
A84 Blood pump according to paragraph A83, wherein the stiffening member protrudes in an axial direction, wherein the potting material stiffens the film-like portion.
A85 Blood pump according to paragraph A83 or A84, wherein the potting material surrounds the at least one stiffening member.
A86 Blood pump according to any one of the preceding paragraphs A83 to A85, wherein the at least one stiffening member is concentric with the axis of rotation of the impeller.
A87 Blood pump according to any one of the preceding paragraphs A1 to A86, wherein the pump housing comprises a drive unit casing, wherein the impeller has at least one primary blade configured to establish a primary blood flow and wherein the impeller has a casing-side end, wherein the casing-side end points to the drive unit casing, and wherein a plurality of secondary blades are provided on the casing-side end configured to establish a secondary blood flow.
A88 Blood pump according to paragraph A87, wherein the impeller has an axis of rotation and wherein each of the secondary blades extends non-radially relative to the axis of rotation.
A89 Blood pump according to paragraph A87 or A88, wherein each of the secondary blades has a base point and an end point, wherein the base points are located on a base circle and the end points are located on an end circle, wherein the base circle and the end circle are concentric with a common center point, and wherein a straight line connecting the base point and the end point of any one of the secondary blades is not running through the center point.
A90 Blood pump according to paragraph A89, wherein each of the plurality of secondary blades is curved relative to the respective straight line.
A91 Blood pump according to any one of the preceding paragraphs A1 to A90, wherein the pump housing comprises a catheter attachment portion, wherein the catheter attachment portion comprises a threading structure on the outer peripheral surface configured to threadedly engage a helical member of a catheter of the blood pump.
A92 Blood pump according to paragraph A91, wherein the blood pump comprises a catheter with a helical member, wherein the catheter is attached to the catheter attachment portion in that the helical member threadedly engages the threading structure, and wherein the helical member is preferably a Nitinol-coil.

As utilized herein, the terms “approximately”, “about”, “substantially” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described without restricting the scope of these features to the precise numerical ranges provided. Accordingly, these terms should be interpreted as indicating that insubstantial or inconsequential modifications or alterations of the subject matter described and are considered to be within the scope of the disclosure. The terms “at least partially” or “partially” as used herein mean both partial and entirely or complete respectively.

### LIST OF REFERENCE SIGNS

10 blood pump
12 pump housing
14 blood flow inlet
16 blood flow outlet
18 passage
20 impeller
22 drive unit casing
24 impeller casing
26 drive unit
28 cannula attachment portion
30 catheter
32 catheter attachment portion
34 tubular portion of catheter attachment portion
36 threading structure
38 helical member/Nitinol-coil
40 first bearing/pivot bearing
42 second bearing/radial bearing
44 bearing accommodation portion
46 crown
48 central tubular portion of crown
50 connecting arms
52 magnet
54 primary blade
56 main body of impeller
58 opening
60 inlet
62 casing-side end of impeller
64 secondary blade
65 secondary auxiliary blade
66 stator
68 insulation assembly
70 post
72 coil winding
74 control/printed circuit board
76 backplate
78 catheter-side end of stator
80 backbone
82 backbone leg
84 spacer
86 frontplate
88 frontsheet
90 spacer assembly
92 tubular portion of spacer
94 central portion of frontplate
96 outer portion of frontplate
98 frontplate leg
100 impeller-side end of stator
102 impeller supporting portion
104 tubular member of impeller supporting portion
106 film like portion
108 protruding pin
110 connecting portion
112 transition portion
114 potting material
116 stiffening member
118 first bearing member/first pivot bearing member
120 second bearing member/second pivot bearing member
122 first abutment portion
124 spherical portion
126 ball
128 support element
130 fitting pin
132 second abutment portion
134 first spherical cap/first calotte
136 second spherical cap/second calotte
138 first cut out
140 second cut out
142 first hollow portion
144 first cooling member
146 second hollow portion
148 second cooling member
149 slot
150 first radial bearing member
152 second radial bearing member
154 collar
156 third cut out
158 impeller cut out
160 fourth cut out
X axis of rotation
CA center axis of opening
CCP common center point
BP base point
BC base circle
EP end point
EC end circle
G gap
SL straight line

1. Blood pump, in particular intravascular blood pump, comprising:
a pump housing having a blood flow inlet and a blood flow outlet connected by a passage,
an impeller disposed in said pump housing, and
a drive unit configured to drive the impeller;
wherein the pump housing comprises a drive unit casing and wherein the drive unit is disposed within the drive unit casing,
wherein the drive unit comprises a stator, and an insulation assembly, and
wherein the insulation assembly is configured to prevent electrical leakage.

2. Blood pump according to claim 1, wherein the insulation assembly comprises a spacer assembly configured to space the stator from the drive unit casing so as to inhibit contact between the stator and the drive unit casing.

3. Blood pump according to claim 2, wherein
the spacer assembly comprises a spacer configured to radially space the stator from an inner surface of the drive unit casing, in particular an inner peripheral surface of the drive unit casing.

4. Blood pump according to claim 3, wherein
the stator has a catheter-side end pointing away from the impeller, wherein the spacer is ring-shaped and has tubular portion, the tubular portion extending at least partially circumferentially,
wherein the spacer is disposed at the catheter-side end of the stator so that the stator is partially disposed radially inside of the tubular portion of the spacer, wherein the tubular portion of the spacer abuts against the inner surface of the drive unit casing, wherein
the stator preferably comprises a backplate contacting the catheter-side end of the stator, and wherein
wherein the backplate is preferably disposed radially inside of the tubular portion of the spacer.

5. Blood pump according to claim 4, wherein
the drive unit further comprises a printed circuit board, wherein the printed circuit board is at least partially disposed radially inside of the spacer assembly.

6. Blood pump according to claim 5, wherein the insulation assembly comprises a frontplate configured to space the stator from the drive unit casing so as to inhibit contact between the stator and the drive unit casing.

7. Blood pump according to claim 6, wherein
the stator comprises a plurality of posts, and coil windings disposed about the posts,
wherein the frontplate has a central portion and an outer portion,
wherein a plurality of frontplate legs extend between the central portion and the outer portion.

8. Blood pump according to claim 7, wherein
the frontplate legs are configured to circumferentially space each of the plurality of posts from an adjacent post, and
wherein the plurality of posts are partially disposed radially inwardly of the outer portion, wherein the outer portion is preferably ring-shaped.

9. Blood pump according to claim 6, wherein
the stator comprises a plurality of posts, and coil windings disposed about the posts.

10. Blood pump according to claim 9, wherein
the backplate and the plurality of posts are integrally formed as a one-piece unitary member.

11. Blood pump according to claim 10, wherein
the stator comprises a backbone having radially extending backbone legs, wherein the backbone legs are preferably configured to circumferentially space each of the plurality of posts from an adjacent post.

12. Blood pump according to claim 11, wherein
the insulation assembly comprises a plurality of shrink elements wherein each of the plurality of posts is at least partially surrounded by one of the shrink elements.

13. Blood pump according to claim 12, wherein
the stator has an impeller-side end pointing towards the impeller,
wherein the insulation assembly further comprises a frontsheet, and
wherein the frontsheet covers the impeller-side end of the stator so as to inhibit contact between the stator and the drive unit casing.

14. Blood pump according to claim 13, wherein
the insulation assembly is at least partially composed of a non-electrical conductive material, wherein the insulation assembly is preferably entirely composed of the non-electrical conductive material, wherein
the non-electrical conductive material preferably is a thermoplastic material, preferably a polyaryletherketone, wherein the non-electrical conductive material is preferably polyetheretherketone.

15. Blood pump according to claim 14, wherein
an inner surface of the drive unit casing is at least partially coated with a non-electrical conductive coating, in particular diamond-like carbon, and/or wherein the stator is at least partially surrounded by a shrink element circumferentially and/or the stator is at least partially coated with a non-electrical conductive coating, and/or wherein
the plurality of posts and/or the backplate and/or the backbone are partially or entirely coated with a non-conductive material, in particular diamond-like carbon (DLC).
